(19) United States
(12) Patent Application Publication
(10) Pub. No.: US 2023/0172498 A1
(43) Pub. Date: Jun. 8, 2023

(54) OPTICAL SYSTEM AND METHOD FOR DETECTING LIGHT SCATTERED FROM TISSUE

(71) Applicant: BAR-ILAN UNIVERSITY, Ramat Gan (IL)

(72) Inventors: Dror FIXLER, Ganei-Tikva (IL); Hamootal DUADI, Givat Shmuel (IL); Idit FEDER, Petach Tikva (IL)

(21) Appl. No.: 17/920,338

(22) PCT Filed: Apr. 22, 2021
(86) PCT No.: PCT/IL2021/050463

§ 371 Date: Oct. 20, 2022

Related U.S. Application Data
(60) Provisional application No. 63/013,720, filed on Apr. 22, 2020.

(51) Int. Cl. A61B 5/1455; A61B 5/00; A61B 5/024

(52) U.S. Cl. CPC A61B 5/14552; A61B 5/7257; A61B 5/02427; A61B 2560/0223

(57) ABSTRACT

A system for detecting light scattered from a tissue and for finding an IPL point for extracting oxygen saturation and pulse rate comprises: (a) at least one light source for illuminating a tissue, the at least one light source has a beam alignable to pass through the tissue; and (b) a plurality of photodetectors/cameras placed at multiple angles with respect to the tissue for collecting the light scattered from the tissue at multiple angles at the same time. The beam of the light source is centered either on a first axis parallel to the tissue and/or on a second axis with respect to the tissue, and the plurality of the photodetectors/cameras are either stationary or movable for conducting measurements at multiple angles for producing a first full scattering profile (FSP) and a second FSP applicable or finding the IPL point for extracting the oxygen saturation and the pulse rate.

### FIELD OF THE INVENTION

The present invention relates to non-invasive optical systems and methods. More particularly, the present invention relates to non-invasive optical systems and methods used in the field of medical science.

### BACKGROUND OF THE INVENTION

Light interacted with tissue is perturbed in a manner that can be detected and quantified by optical set-ups coupled with mathematical light transport models. Such information can be employed to assess the health of tissue and its characteristics. Thus, over the years and particularly since the laser has been developed, researchers have developed numerous techniques in which tissue is illuminated with light, having prescribed properties such as wavelength, frequency, coherence, spatial profile, and the portion of this light that returns to the surface is detected and analyzed.

### SUMMARY OF THE INVENTION

The present invention is of an optical system and method for detecting light scattered from tissue. In accordance with some embodiments of the present invention, there is provided

A system for detecting light scattered from a tissue and for finding an IPL point for extracting oxygen saturation and pulse rate comprising:

(i) at least one light source for illuminating a tissue, said at least one light source having a beam alignable to pass through the tissue; and

(ii) a plurality of photodetectors/cameras placed at multiple angles with respect to the tissue for collecting the light scattered from the tissue at multiple angles at the same time;

wherein the beam of said light source is centered either on a first axis parallel to the tissue and/or on a second axis with respect to the tissue, and

the plurality of said photodetectors/cameras are either stationary or movable for conducting measurements at multiple angles;

thereby, for producing a first full scattering profile (FSP) and a second FSP applicable for finding the IPL point for extracting the oxygen saturation and the pulse rate.

In accordance with some embodiments of the present invention, the light source is a continuous wave laser selected from a He—Ne gas laser, a Ti:sapphire laser, and a GaAlAs laser.

In accordance with some embodiments of the present invention, the photodetectors are selected from fixed gain silicon-type detectors and Gallium Arsenide type-detectors.

In accordance with some embodiments of the present invention, each one of said photodetectors has an active area ranging between 0.1 mm² and 10 mm².

In accordance with some embodiments of the present invention, the photodetectors/cameras are positioned successively in increments ranging between 2 to 10 degrees.

In accordance with some embodiments of the present invention, the above-described system is applied for at least one of PPG signal extraction, blood oxygen saturation measurement, pulse rate measurement, blood pressure measurement, respiratory rate measurement, perfusion and blood sugar level detection.

In accordance with some embodiments of the present invention, the system is applied for extraction properties of scattering liquids in order to assess the quality of said scattering liquids.

In accordance with some embodiments of the present invention, wherein the scattering liquids are selected from oil, petroleum, water, and wine.

In accordance with some embodiments of the present invention, there is provided a method for detecting light scattered from tissue for extracting light intensity at an iso-pathlength (IPL) point. The method comprising the following steps:
(A) providing the above system;
(B) using either solid or liquid phantoms to calibrate said system;
(C) conducting measurements on tissue by illuminating the tissue with both a light beam centered on a first axis parallel to the tissue for producing a first full scattering profile (FSP) and on a second axis;
   for producing a second full scattering profile (FSP);
(D) examining said first FSP and said second FSP and locating an IPL point;
   and
(E) extracting light intensity at the IPL point.

In accordance with some embodiments of the present invention, the above method is used for extracting oxygen saturation and comprises the following steps:
extracting light intensity at a second point, at 9=0; and
deriving an oxygen saturation expression, S, and determining an oxygen saturation value by:

using I_{μ}=I₀exp(−εcl) equation (3)
wherein
   I₀ is the light intensity without absorption;
   I is optical path length that depends on the scattering;
   ε is the extinction coefficient; and
   c is the concentration of blood;
   for a given wavelength,

I_{μ}(θ)=I₀(θ)exp(−[S·ε_{HbO2}+(1−S)·ε_{Hb}]c·l(μ′ₛ)) equation (5)
at the IPL point, the optical path length, l, is constant, thus,

I_{μ}(θ_{IPL})=I₀(θ)exp(−[S·ε_{HbO2}+(1−S)·ε_{Hb}]c·l) equation (6)
calculating an average optical path value via

$$l = D sin ( \frac{180 - {θ}_{IPL}}{2} ) \cdot DPF \qquad equation ( 7 )$$

wherein
D is the diameter of the tissue; and
DPF is a differential pathlength factor;
taking the natural log on equation (6) and using equation (7) to derive:

$$\frac{ln [ {I}_{μ} ( {θ}_{IPL} ) / {I}_{0} ( {θ}_{IPL} ) ]}{cD sin ( \frac{180 - {θ}_{IPL}}{2} ) D P F} = S ( {ℰ}_{H b} - {ℰ}_{HbO 2} ) - {ℰ}_{H b} \qquad equation ( 8 )$$

Using the source intensity, Iₛ, to asses I₀

I₀(θ_{IPL})=K·Iₛ equation (9)
substituting Jo in equation (8):

$$\frac{In [ {I}_{μ} ( {θ}_{IPL} ) / ( {I}_{s} K ) ]}{cD sin ( \frac{180 - {θ}_{IPL}}{2} ) D P F} = S ( {ℰ}_{H b} - {ℰ}_{HbO 2} ) - {ℰ}_{H b} \qquad equation ( 10 )$$

and extracting oxygen saturation expression, S, from equation (10) and calculating an oxygen saturation value.

In accordance with some embodiments of the present invention, the above method further comprising calculating the standard deviation of said oxygen saturation value.

In accordance with some embodiments of the present invention, the above method further comprises extracting pulse rate via the following steps:
generating a Photoplethysmogram (PPG) profile at the IPL point;
performing a Fourier transform on the PPG profile;
passing the Fourier transformed PPG profile through a bandpass filter;
extracting a maximum frequency value, fₘₐₓ; and
converting said maximum frequency value, fₘₐₓ, to pulse rate according to pulse rate=fₘₐₓ*60.

In accordance with some embodiments of the present invention, the above method further comprises calculating the standard deviation of the calculated pulse rate.

In accordance with some embodiments of the present invention, the above-described method is applied for at least one of PPG signal extraction, blood oxygen saturation measurement, pulse rate measurement, blood pressure measurement, respiratory rate measurement, perfusion and blood sugar level detection.

In accordance with some embodiments of the present invention, the method is applied for extraction properties of scattering liquids in order to assess the quality of said scattering liquids, the scattering liquids are selected from oil, petroleum, water, and wine.

### BRIEF DESCRIPTION OF THE FIGURES

FIGS. 1A&B are schematics of a measurement system used for detecting light scattered from tissue for extracting pulse rate and oxygen saturation in accordance with some embodiments of the present invention.

FIG. 2A illustrates solid phantoms and the corresponding full scattering profiles. FIG. 2B illustrates full scattering profiles (FSPs) of 13 mm diameter cylindrical solid phantoms with scattering properties and without absorbing components.

FIGS. 3A&B illustrate the dependency of the iso-pathlength (IPL) point on the radius of phantom and/or human fingers.

FIG. 3C illustrates absorption coefficients extracted from phantom measurements (dots) in comparison to theoretical absorption coefficients (line) in two different diameters: 10 mm (circle) and 13 mm (triangles).

FIG. 4 is a cross-sectional illustration of a cylindrical tissue in accordance with some embodiments of the present invention.

FIG. 5 summarizes the steps of a method used for determining the oxygen saturation in human tissue in accordance with some embodiments of the present invention.

FIG. 6 summarizes the steps of a method used for determining the pulse rate in accordance with some embodiments of the present invention.

### DETAILED DESCRIPTION OF THE FIGURES

The present invention is of an optical measurement system and method for detecting light scattered from tissue.

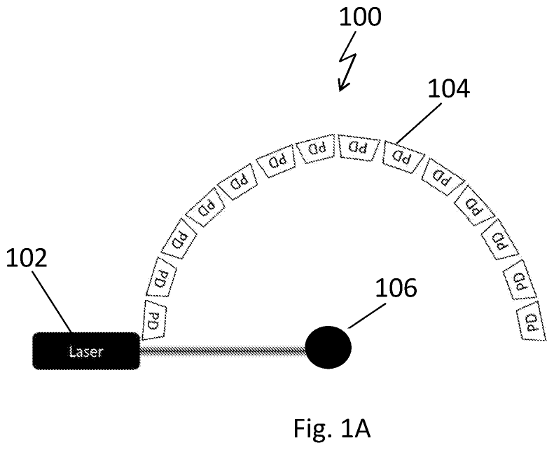

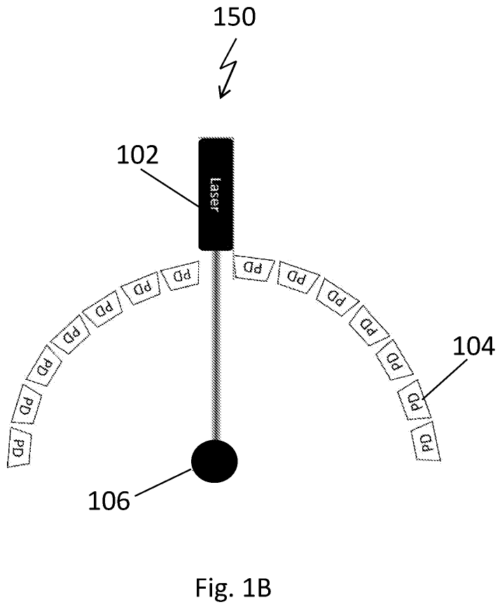

FIGS. 1A&B are schematics of a measurement system 100, having two configurations 100A&B, which are used for measuring light scattered from tissue at multiple angles in accordance with some embodiments of the present invention. As seen in the figures, measurement system 100 comprises a continuous wave laser 102 for illuminating a sample and a plurality of photodetectors 104 for collecting the light scattered from the sample simultaneously.

Continuous wave laser 102 may be selected from, but not limited to, a He—Ne gas laser, a Ti:sapphire laser, and a GaAlAs laser with an excitation wavelength in the near-infrared regime, i.e., 633 nm, 650 nm, 660 nm, 785 nm, 850 nm or 880 nm and a maximum power of 5 mW.

In accordance with some embodiments of the present invention, photodetectors 104 may be selected from, but not limited to, fixed gain silicon-type detectors, and Gallium Arsenide type-detectors. Photodetectors 104 may have an active area of 0.1 mm² or more and may be positioned every ten degrees or less in close proximity to the tissue to overcome light scattering and thus to improve the light collection efficiency.

FIGS. 1A&B illustrate two optional positions of laser 102 in accordance with some embodiments of the present invention. In FIG. 1A, laser 102 is positioned on a first axis (the major axis) so that its beam aligned with an axis parallel to the tissue, and photons, entering and passing through the tissue from the major axis, are detected via photodetectors/cameras 104 at multiple angles at the same time.

In FIG. 1B laser 102 is positioned on a second axis (the minor axis) so that its beam is aligned with an axis normal to the tissue, and photons entering and passing through the tissue from the minor axis are detected via photodetectors/cameras 104 at multiple angles at the same time.

In accordance with some embodiments of the present invention, the laser 102 may be kept stationary with its beam aligned either on a first axis parallel to the tissue and/or on a second axis with respect to the tissue, while the photodetectors/cameras 104 are movable (alternatively, additional photodetectors are used) for conducting measurements at multiple angles.

It should be noted that optical properties are not constant and vary at various conditions, for instance, while breathing. Thus, in accordance with some embodiments of the present invention, the use of an array of photodetectors 104 for conducting measurements at multiple angles at the same time is highly essential for producing accurate full scattering profiles (FSP), and thus, for extracting pulse rate and/or calculating oxygen saturation values that are highly accurate and robust.

Calibrating the System

In accordance with some embodiments of the present invention, measurement system 100 is calibrated with cylindrical tubes filled with tissue-like solid phantoms which mimic the structure and optical properties of tissues such as human fingers where each of the phantoms having different optical properties, i.e., different scattering and absorption coefficients.

Such tissue-like solid phantoms may be prepared using 1% Agarose powder for solidification and varying concentrations of scattering components such as Intralipid (IL, Lipofundin MCT/LCT 20%, B. Braun Melsungen AG, Germany) where the Intralipid concentration, x[%], is a function of the desired reduced scattering coefficient, μₛ′ (in units of cm⁻¹), of each phantom and is calculated as follows:

x[%]=0.89*μₛ′−0.1531

Additionally, tissue-like solid phantoms may be prepared using varying concentrations of an absorbing component such as, for instance, India ink 0.1%, for varying the degree of absorption from 0.01 cm⁻¹ to 14 cm⁻¹. Based on the desired absorption coefficient, μ_{a,} of each phantom, the concentration y[%] of the absorbing component is calculated as follows:

y[%]=1.8e⁻³*μₐ

Thus, in accordance with some embodiments of the present invention, measurement system 100 is calibrated as follows: tissue-like solid phantom is illuminated by a single laser source, laser 102 positioned on either the major or the minor axis (seen in FIG. 1A), and a full scattering profile (FSP) is detected by multiple photodetectors 104.

The scattering profiles are compared for extracting the iso-pathlength (IPL) point which is a point at which the intensity of light is identical.

Phantom Experimental Results

In accordance with some embodiments of the present invention, measurement system 100 of FIGS. 1A&B may be used for illuminating cylindrical solid phantoms with scattering properties and with and without absorbing components in order to examine the influence of absorption on the full scattering profile.

In accordance with some embodiments of the present invention, measurement system 100 is used for generating scattering profiles of cylindrical solid phantoms such as 10 mm diameter cylindrical solid phantoms having same scattering coefficient, for instance, a scattering coefficient of 20 cm⁻¹ and various absorption coefficients.

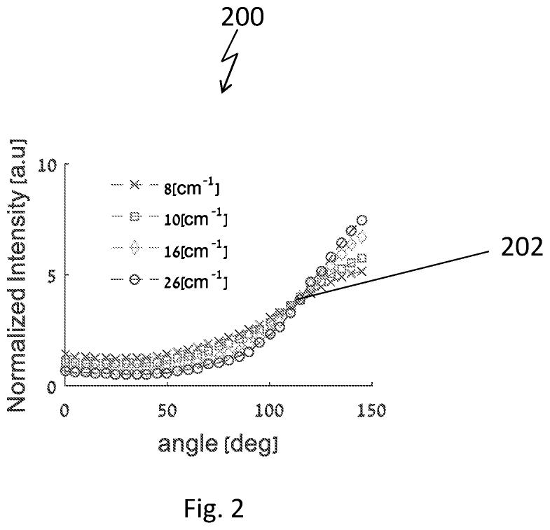

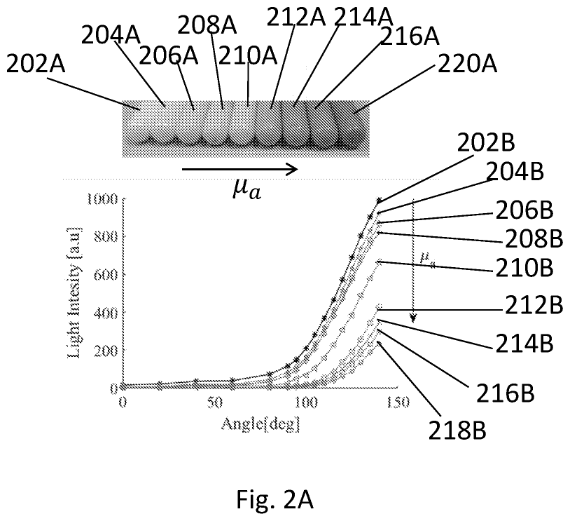

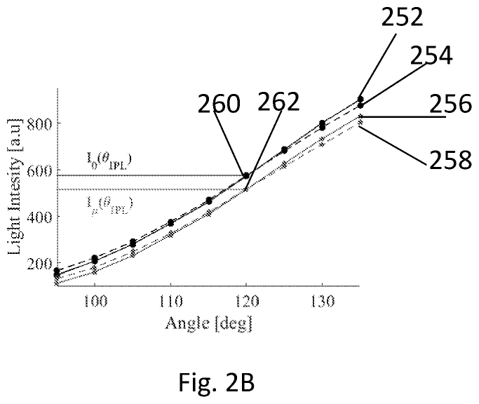

FIG. 2A illustrates solid phantoms 202A-218A and the corresponding full scattering profiles (FSPs) 202B-218B. Each curve is a scattering profile of a single phantom-curve 202B is a full scattering profile of phantom 202A without absorbing components, and curves 204B-218B are full scattering profiles of phantoms 204A-218A with absorbing components, e.g., absorption coefficients ranging from 0.013 cm⁻¹ to 0.36 cm⁻¹.

As seen in the figure, scattering profiles 202B-218B are all same in shape, however, the intensity decreases as the absorption increases.

In order to find a point at which the light intensity is identical, i.e., the IPL point, measurement system 100 may be used for illuminating cylindrical solid phantoms with and without absorbing components. The illumination is carried out in two perpendicular orientations for producing a full scattering profile in each one of the orientations, and the scattering profiles are then compared for extracting the IPL point.

FIG. 2B illustrates full scattering profiles (FSPs) of 13 mm diameter cylindrical solid phantoms with scattering components and with/without absorbing components. The scattering profiles were generated via illumination carried out in two perpendicular orientations.

Curves 252 and 254 are full scattering profiles without absorption while curves 256 and 258 are full scattering profiles with absorption, e.g., an absorption coefficient of 0.13 cm⁻¹.

Curves 252 and 256 correspond to a scattering coefficient of 20 cm⁻¹ and curves 254 and 258 correspond to a scattering coefficient of 26 cm⁻¹.

Seen in the figure, are the IPL points, IPL point 260 and IPL point 262, which appear to be at the same angle in both profiles, e.g., in the scattering profile with absorption and in the scattering profile without absorption.

Also seen in the figure, the light intensity at the IPL point without absorption (I₀(θ_{IPL})) is higher than the intensity at the IPL point with absorption (I_{μ}(θ_{IPL})).

In accordance with some embodiments of the present invention, the curves in FIG. 2B are used for calibrating K and calculating DPF₀.

K is calculated by equation (1):

K=I₀/Iₛ equation (1)

and the DPF₀ is calculated by equation (2):

$$D P {F}_{0} = \frac{\sqrt{{I}_{0} ( {θ}_{IPL} ) / {I}_{μ} ( {θ}_{IPL} )}}{{μ}_{a} D sin ( \frac{180 - {θ}_{IPL}}{2} )} ln ( \frac{{I}_{0} ( {θ}_{IPL} )}{{I}_{μ} ( {θ}_{IPL} )} ) \qquad equation ( 2 )$$

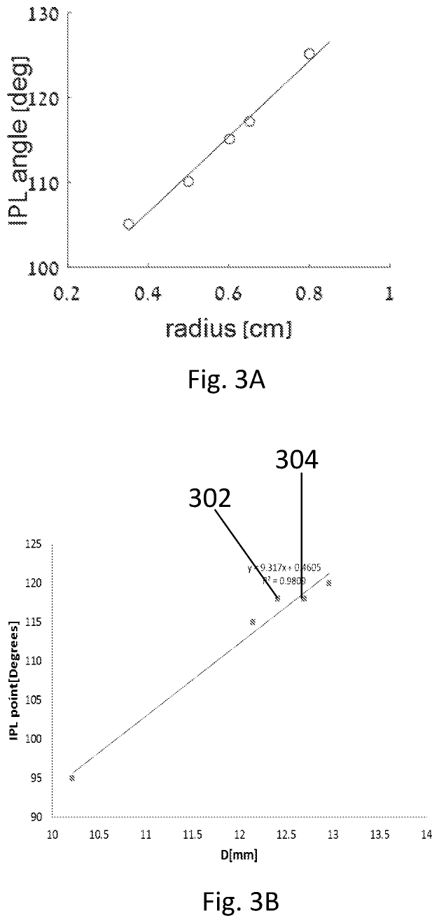

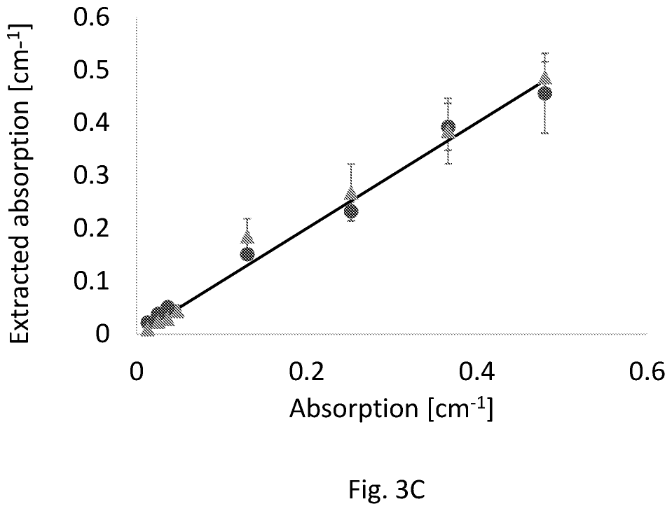

FIGS. 3A&B illustrate the dependency of the IPL point on the radius/diameter of tissue-like liquid phantoms and human fingers respectively. As seen in the figures, the position of the IPL point is linearly related to the effective radius/diameter of the finger; the position of the IPL point increases as the radius/diameter of the finger increases. Seen in FIG. 3B points 302 and 304 represent measurements of two different people.

FIG. 3C illustrates absorption coefficients extracted from phantom measurements (dots) in comparison to theoretical absorption coefficients (line) in two different diameters: 10 mm (circle) and 13 mm (triangles). Based on the IPL point at 105 degrees and 120 degrees respectively.

In accordance with some embodiments of the present invention, the experimental absorption coefficients shown in FIG. 3C are calculated via a set of equations (described below) using experimental parameters which are extracted from FIGS. 2A and 2B.

In Vivo Fsp Measurements

In accordance with some embodiments of the present invention, measurement system 100 is used for illuminating tissue, such as a human finger, in two perpendicular orientations for producing a full scattering profile in each one of the orientations. Such scattering profiles are then compared for extracting a point at which the light intensity is identical, i.e., the IPL point and for generating a full Photoplethysmogram (PPG) profile.

A PPG profile is intensity vs. time profile of light scattered from tissue. Such a profile is used for detecting changes in blood volume in the finger, i.e., for monitoring heart rate and a cardiac cycle of a patient.

Determination of Oxygen Saturation

In accordance with some embodiments of the present invention, the intensities of light scattered from a finger at two angles (a) at the IPL point and (b) at a relatively small angle and preferably at an angle as small as 0° are measured and used for calculating the oxygen saturation.

It should be noted that the intensity measured at the IPL point with the intensity measured at a relatively small angle neutralizes any variation in the intensity of the laser beam.

Based on the above measurements, the oxygen saturation is calculated as follows:

according to the Beer-Lambert law,

I_{μ}=I₀exp(−εcl) equation (3)

where

I₀ is the light intensity without absorption;

I is the optical path length that depends on the scattering;

ε is the extinction coefficient; and

c is the concentration of blood.

The extinction coefficient s, is a function of the extinction coefficients of Hb (ε_{Hb}) and HbO₂ (ε_{HbO2}) and the saturation value, S, as follows:

ε=S·ε_{HbO2}+(1−S)·ε_{Hb} equation (4)

In the case of a cylindrical-shaped tissue, such as a human finger, the optical path length, l, and the extinction coefficient, ε, depend on the wavelength of the scattered light at all angles with the exception of the IPL point. More specifically, the optical path length, l, depends on the reduced scattering coefficient, μₛ′, which depends on the wavelength.

Thus, for a given wavelength,

I_{μ}(θ)=I₀(θ)exp(−[S·ε_{HbO2}+(1−S)·ε_{Hb}]c·l(μ′ₛ)) equation (5)

At the IPL point, the optical path length, l, is constant, yielding:

I_{μ}(θ_{IPL})=I₀(θ)exp(−[S·ε_{HbO2}+(1−S)·ε_{Hb}]c·l) equation (6)

The average optical path length, l, is not measured directly but is calculated by equation (7).

$$l = D sin ( \frac{180 - {θ}_{IPL}}{2} ) \cdot DPF \qquad equation ( 7 )$$

$$where$$

$$DPF = DP {F}_{0} \sqrt{{I}_{μ} ( {θ}_{IPL} ) / {I}_{0} ( {θ}_{IPL} )} \qquad equation ( 8 )$$

and where

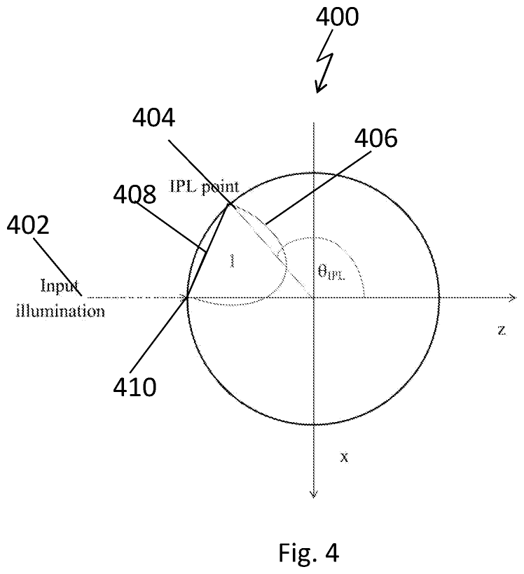

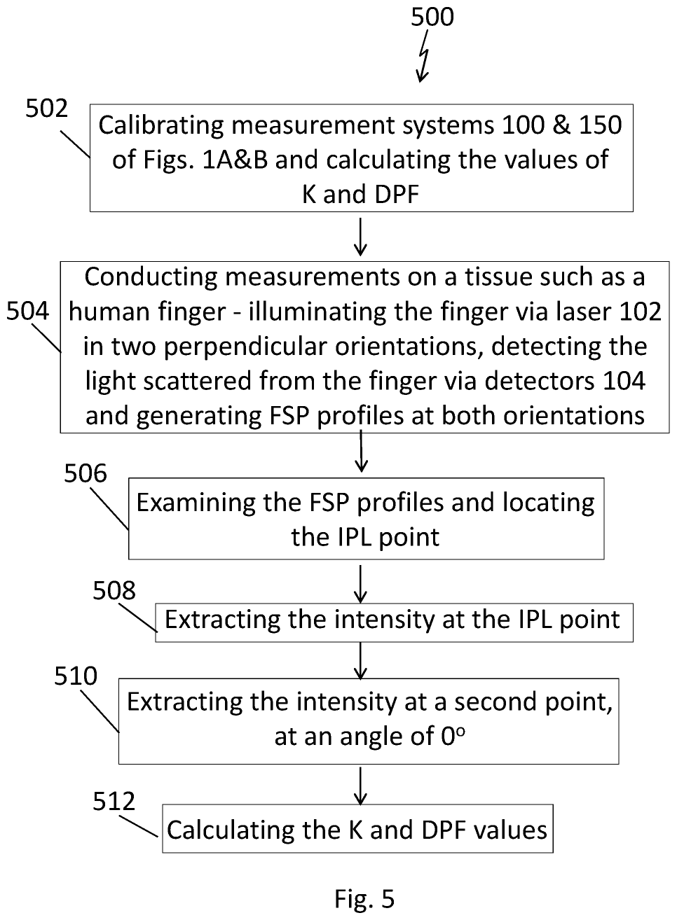

D sin ((180−θ_{IPL})/2) is the length of segment 408 seen in FIG. 4,

D is the diameter of the finger, and

DPF is a differential path length factor, correcting the length of segment 408 in FIG. 4

FIG. 4 is a cross-sectional illustration of a cylindrical tissue 400 in accordance with some embodiments of the present invention.

As seen in the figure, laser beam 402 enters the cross-section of circular tissue 400 in the z-direction, and optical path length, l, 406 is the average path length of photons propagating through the tissue.

Taking the natural log on equation (6) and using equation (7) we derive:

$$\frac{ln [ {I}_{μ} ( {θ}_{IPL} ) / {I}_{0} ( {θ}_{IPL} ) ]}{cD sin ( \frac{180 - {θ}_{IPL}}{2} ) D P F} = S ( {ℰ}_{H b} - {ℰ}_{HbO 2} ) - {ℰ}_{H b} \qquad equation ( 9 )$$

Since I₀ is unknown, the source intensity Iₛ is used, to asses I₀

I₀(θ_{IPL})=K·Iₛ equation (10)

Substituting I₀(θ_{IPL}) in Eq. (9) leads to:

$$\frac{ln [ \frac{{I}_{μ} ( {θ}_{IPL} )}{( {I}_{s} K )} ]}{cD sin ( \frac{180 - {θ}_{IPL}}{2} ) D P F} = S ( {ℰ}_{H b} - {ℰ}_{HbO 2} ) - {ℰ}_{H b} \qquad equation ( 11 )$$

Equation (11) is used for extracting the oxygen saturation, S, an expression where the value of the K and DPF₀ constants are calculated from calibration measurements.

The initial light intensity of the laser, Iₛ, is measured and K is calculated from calibration measurements using solid phantoms with scattering properties and no absorbing components, where K is dependent on the diameter. K is calculated for θ_{IPL} according to equation (1) as follows:

K=I₀(θ_{IPL})/Iₛ equation (12)

The DPF₀ expression is calculated from measurements using solid phantoms with scattering and absorption properties.

Let I₀(θ_{IPL}) be the intensity at the IPL angle for a phantom with no absorbing component, and I_{μ}(θ_{IPL}) the intensity at the IPL angle for a phantom with an absorbing component (absorption of μₐ). According to Beer-Lambert law, equation (3), the intensity at the IPL point is:

$${I}_{μ} ( {θ}_{IPL} ) = {I}_{0} ( {θ}_{IPL} ) exp ( - {μ}_{a} l ) = {I}_{0} ( {θ}_{IPL} ) exp \{ - {μ}_{a} D sin ( \frac{180 - {θ}_{IPL}}{2} ) D P F \} \qquad equation ( 13 )$$

Hence, the DPF₀ is calculated via equation (2):

$$D P {F}_{0} = \frac{\sqrt{{I}_{0} ( {θ}_{IPL} ) / {I}_{μ} ( {θ}_{IPL} )}}{{μ}_{a} D sin ( \frac{180 - {θ}_{IPL}}{2} )} ln ( \frac{{I}_{0} ( {θ}_{IPL} )}{{I}_{μ} ( {θ}_{IPL} )} ) \qquad equation ( 14 )$$

The obtained values of K and DPF₀, as obtained from the calibration measurements, are used in Eq. (11).

In order to examine the accuracy of equation (11), the oxygen saturation values in the blood of multiple people were calculated by the above-described method. The oxygen saturation values of the same people were also measured via a medical pulse oximetry device and/or by blood tests. Then, equation (15) was used for computing the standard deviation, i.e., for determining the accuracy of the oxygen saturation values calculated by equation (11).

STD=\sqrt{Σ(S_{method}−S_{blood})²} equation (15)

TABLE 1

| | D/2 | Iₜ(θ_{IPL}) | θ_{IPL} | Iₜ(θ = 0) | S |
|---|---|---|---|---|---|
| (a) | 6.07 | 10.25 | 115 | 0.8 | 0.9725 |
| (b) | 6.48 | 11.15 | 125 | 0.8 | 0.9817 |

(a) data obtained by the above-described method, and (b) data obtained with a medical pulse oximetry device.

Seen in Table 1, line (a) provides data obtained by the above-described method; the data was extracted from the FSP profile of a human finger with an effective radius of 6.07 mm, and the saturation value, S, was calculated using equation (11).

Line (b) provides data measured by a medical pulse oximetry device for the purpose of comparison.

Seen in Table 1, the saturation value, S, calculated by equation (11) is comparable to the saturation value measured by a medical pulse oximetry device—the standard deviation, STD, calculated by equation (14) is ±0.15.

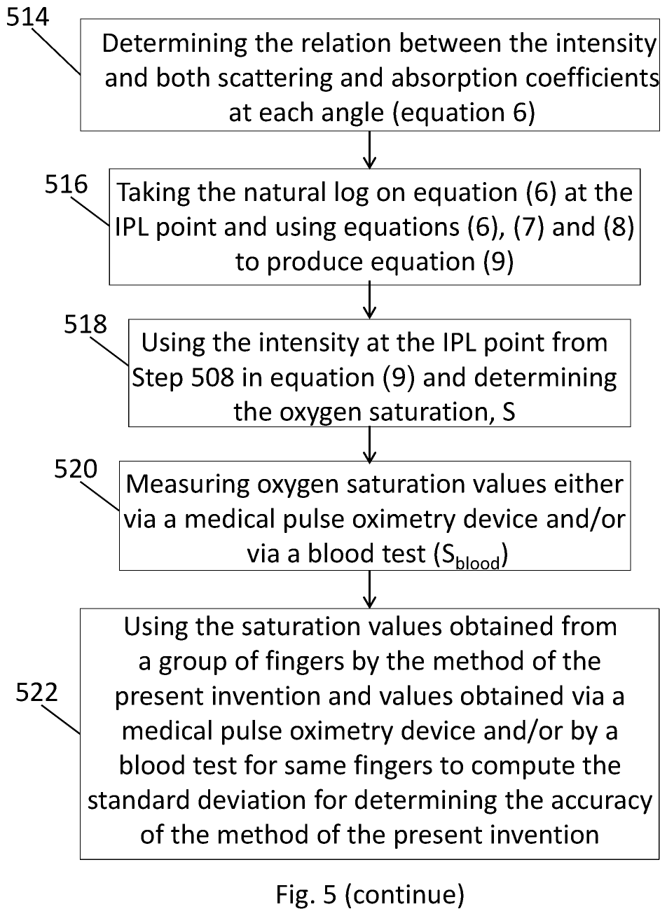

FIG. 5 summarizes the steps of a method used for detecting light scattered from tissue for extracting light intensity at an IPL point and for determining the oxygen saturation in a human tissue 500 in accordance with some embodiments of the present invention. The method comprising the following steps:

Step 502—Calibrating measurement system 100 using the calibration method described above and calculating the values of K and DPF₀;

Step 504—Conducting measurements on tissue such as a human finger—illuminating the finger via laser 102 in two perpendicular orientations, detecting the light scattered from the finger via an array of detectors 104 and generating FSP profiles at both orientations;

Step 506—Examining the FSP profiles and locating the IPL point;

Step 508—Extracting the intensity at the IPL point;

Step 510—Extracting the intensity at a second point, for instance, at θ=0⁰ (full transmission);

Step 512—Calculating DPF value;

Step 514—Determining the relation between the intensity and both scattering and absorption coefficients at each angle (equation 6);

Step 516—Taking the natural log on equation (6) at the IPL point and using equations (6), (7), and (8) to produce equation (9);

Step 518—Using the intensity at the IPL point from step 508 in equation (9) and determining the oxygen saturation, S;

Step 520—Measuring oxygen saturation values either via a medical pulse oximetry device and/or via a blood test (S_{blood});

Step 522—Using the saturation values obtained from a group of fingers by the method of the present invention and values obtained via a medical pulse oximetry device and/or by a blood test for same fingers to compute the standard deviation for determining the accuracy of the method of the present invention.

Determination of Pulse Rate

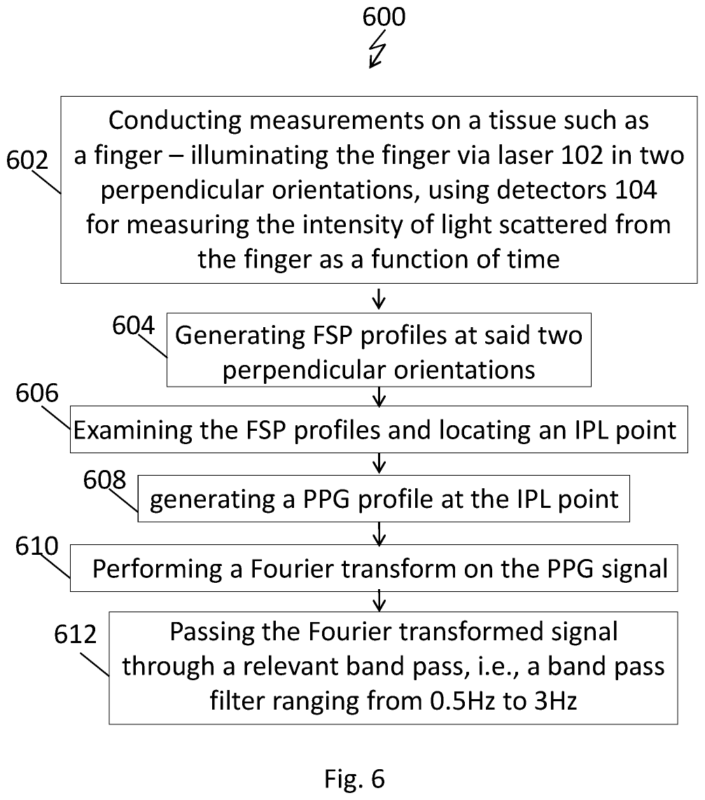

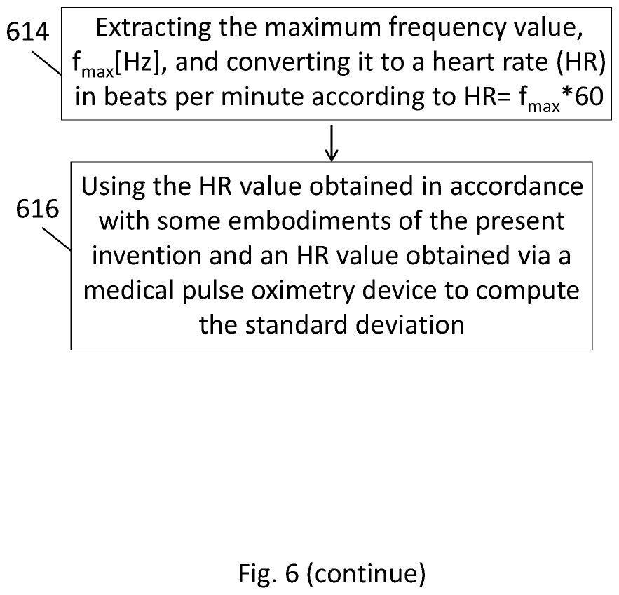

FIG. 6 summarizes the steps of a method 600 used for detecting light scattered from tissue for extracting light intensity at an IPL point and for determining the pulse rate in accordance with some embodiments of the present invention. The method comprising the following steps:

Step 602—Conducting measurements on tissue such as a human finger—illuminating the finger via laser 102 in two perpendicular orientations, using an array of detectors 104 for detecting the light scattered from the finger as a function of time;

Step 604—Generating FSP profiles at said two perpendicular orientations;

Step 606—Examining the FSP profiles and locating an IPL point;

Step 608—Generating a PPG profile at the IPL point;

Step 610—Performing a Fourier transform on the PPG signal;

Step 612—Passing the Fourier transformed signal through a relevant bandpass filter, i.e., through a bandpass filter ranging from 0.5 Hz to 3 Hz;

Step 614—Extracting the maximum frequency value, fₘₐₓ, and converting it to a heart rate (HR) in beats per minute according to HR=fₘₐₓ*60; and

Step 616—Determining the accuracy of the method of the present invention by calculating the standard deviation of the HR value obtained by the method of the present invention and an HR value obtained via a medical pulse oximetry device for same fingers.

It should be noted that the system and the method of the present invention may be applied for extracting a PPG signal, measuring blood oxygen saturation, and measuring pulse rate. In addition, the system and method of the present invention may be applied for measuring blood pressure, measuring respiratory rate, detecting blood sugar level and other biomarkers, and perfusion,

Additionally, the system and method of the present invention may be applied for extracting properties of scattering liquids such as, for instance, oil, petroleum, water, wine and the like in order to assess their quality.

1. A system for detecting light scattered from a tissue and for finding an iso-pathlength (IPL) point for extracting oxygen saturation and pulse rate comprising:
(i) at least one light source for illuminating a tissue, said at least one light source having a beam alignable to pass through the tissue; and
(ii) a plurality of photodetectors placed at multiple angles with respect to the tissue for collecting the light scattered from the tissue at multiple angles at the same time;
wherein the beam of said light source is centered either on a first axis parallel to the tissue and/or on a second axis with respect to the tissue, and
the plurality of said photodetectors are either stationary or movable for conducting measurements at multiple angles;
thereby, for producing a first full scattering profile (FSP) and a second FSP applicable for finding the IPL point for extracting the oxygen saturation and the pulse rate.

2. The system of claim 1, wherein said at least one light source is a continuous wave laser selected from a He—Ne gas laser, a Ti:sapphire laser, and a GaAlAs laser.

3. The system of claim 1, wherein said photodetectors are selected from fixed gain silicon-type detectors and Gallium Arsenide type-detectors.

4. The system of claim 1, wherein each one of said photodetectors has an active area ranging between 0.1 mm² and 10 mm².

5. The system of claim 1, wherein said photodetectors are positioned successively in increments ranging between 2 to 10 degrees.

6. The system of claim 1, is applied for at least one of Photoplethysmogram (PPG) signal extraction, blood oxygen saturation measurement, pulse rate measurement, blood pressure measurement, respiratory rate measurement, perfusion and blood sugar level detection.

7. The system of claim 1, applied for extraction properties of scattering liquids in order to assess the quality of said scattering liquids.

8. The system of claim 7, wherein said scattering liquids are selected from oil, petroleum, water, and wine.

9. A method for detecting light scattered from tissue for extracting light intensity at an iso-pathlength (IPL) point:
(A) providing a system according to claim 1;
(B) using either solid or liquid phantoms to calibrate said system;
(C) conducting measurements on tissue by illuminating the tissue with both a light beam centered on a first axis parallel to the tissue for producing a first full scattering profile (FSP) and on a second axis for producing a second full scattering profile (FSP);
(D) examining said first FSP and said second FSP and locating an iso-pathlength (IPL) point; and
(E) extracting light intensity at the IPL point.

10. The method of claim 9 for extracting oxygen saturation further comprising:
extracting light intensity at a second point, at θ=0; and
deriving an oxygen saturation expression, S, and determining an oxygen saturation value using
I_{μ}=I₀exp(−εcl)
wherein I₀ is the light intensity without absorption, l is optical path length that depends on the scattering, ε is the extinction coefficient, and
c is concentration of blood, for a given wavelength,
wherein I_{μ}(θ)=I₀(θ)exp(−[S·ε_{HbO2}+(1−S)·ε_{Hb}]c·l(μ′ₛ))
at the IPL point, wherein the optical path length, l, is constant, and thus,
I_{μ}(θ_{IPL})=I₀(0)exp(−[S·ε_{HbO2}+(1−S)·ε_{Hb}]c·l)
calculating an average optical path value via
$$l = D sin ( \frac{180 - {θ}_{IPL}}{2} ) \cdot DPF$$
wherein D is the diameter of the tissue, and wherein DPF is a differential pathlength factor;
taking the natural log of I_{μ}(θ_{IPL})
using
$$l = D sin ( \frac{180 - {θ}_{IPL}}{2} ) \cdot DPF$$
to derive:
$$\frac{ln [ {I}_{μ} ( {θ}_{IPL} ) / {I}_{0} ( {θ}_{IPL} ) ]}{cD sin ( \frac{180 - {θ}_{IPL}}{2} ) D P F} = S ( {ℰ}_{H b} - {ℰ}_{HbO 2} ) - {ℰ}_{H b} :$$
using Iₛ to assess I₀ using:
I₀(θ_{IPL})=K·Iₛ
using I₀ in
$$\frac{In [ {I}_{μ} ( {θ}_{IPL} ) / ( {I}_{s} K ) ]}{cD sin ( \frac{180 - {θ}_{IPL}}{2} ) D P F} = S ( {ℰ}_{H b} - {ℰ}_{HbO 2} ) - {ℰ}_{H b}$$
and
extracting oxygen saturation expression, S, from
$$\frac{In [ {I}_{μ} ( {θ}_{IPL} ) / ( {I}_{s} K ) ]}{cD sin ( \frac{180 - {θ}_{IPL}}{2} ) D P F} = S ( {ℰ}_{H b} - {ℰ}_{HbO 2} ) - {ℰ}_{H b}$$
and calculating an oxygen saturation value.

11. The method of claim 10, comprising calculating the standard deviation of said oxygen saturation value.

12. The method of claim 9 for extracting pulse rate, comprising:
generating a Photoplethysmogram (PPG) profile at the IPL point;
performing a Fourier transform on the PPG profile;
passing the Fourier transformed PPG profile through a bandpass filter;
extracting a maximum frequency value, fₘₐₓ; and
converting said maximum frequency value, fₘₐₓ to pulse rate according to pulse rate=fₘₐₓ*60.

13. The method of claim 12 further comprising calculating the standard deviation of the calculated pulse rate.

14. The method of claim 9, is applied for at least one of extracting PPG signal, measuring blood oxygen saturation, measuring pulse rate, measuring blood pressure, measuring respiratory rate, perfusion, and detecting blood sugar level.

15. The method of claim 9, is applied for extracting properties of scattering liquids in order to assess the quality of said scattering liquids.

16. The method of claim 15, wherein said scattering liquids are selected from oil, petroleum, water, and wine.
